(19) United States
(12) Patent Application Publication
(10) Pub. No.: US 2014/0243240 A1
(43) Pub. Date: Aug. 28, 2014

(54) microRNA EXPRESSION PROFILING OF THYROID CANCER

(71) Applicant: Georgetown University, Washington, DC (US)

(72) Inventors: Offie P. Soldin, Washington, DC (US); Yurly Gusev, Washington, DC (US); Catalin Marian, Washington, DC (US)

(21) Appl. No.: 14/354,442

(22) PCT Filed: Oct. 24, 2012
(86) PCT No.: PCT/US12/61560

§ 371 Date: Apr. 25, 2014

Related U.S. Application Data
(60) Provisional application No. 61/551,858, filed on Oct. 26, 2011.

(51) Int. Cl. C12Q 1/68

(52) U.S. Cl. CPC C12Q 1/6886
USPC 506/9

(57) ABSTRACT

Methods of screening an individual for thyroid cancer or a potential for developing thyroid cancer, and for diagnosing thyroid cancer or a potential for developing thyroid cancer in an individual subject in need thereof includes: determining expression levels of at least one miRNA selected from a first group of specific miRNAs; compare the expression levels of the at least one miRNA from the first group with the expression levels of the corresponding miRNA established for a control individual not having thyroid cancer or nodular hyperplasia, and identifying differences in the miRNA expression levels between the at least one sample from said individual subject and miRNA expression levels established for a control individual not having thyroid cancer or nodular hyperplasia. Methods of treating an individual subject having thyroid cancer and/or a potential for developing thyroid cancer using these procedures in selecting the most appropriate method of treatment for the particular stage and/or type of thyroid cancer.

### CROSS REFERENCE TO RELATED APPLICATIONS

This application is a 371 application of International Application No. PCT/US2012/061560, filed Oct. 24, 2012, which claims priority to U.S. Provisional Patent Application No. 61/551,858, filed Oct. 26, 2011, each of which is incorporated by reference in its entirety herein.

### TECHNICAL FIELD

The subject matter described herein relates to the fields of molecular biology and medicine. More specifically, it relates to the field of thyroid cancer research, diagnosis and treatment.

### BACKGROUND

Thyroid cancer is a heterogeneous disease arising from the follicular or medullar cells of the thyroid. There are several types of thyroid cancer including:
Anaplastic carcinoma (also called giant and spindle cell cancer), which is the most dangerous form of thyroid cancer. It is rare, and does not respond to radioiodine therapy. Anaplastic carcinoma spreads quickly.
Follicular carcinoma, which accounts for about 10% of all cases, is more likely to recur and spread.
Medullary carcinoma, which is a cancer of nonthyroid cells that are normally present in the thyroid gland, tends to occur in families and has been linked with several specific genetic mutations. It requires different treatment than other types of thyroid cancer.
Papillary carcinoma, which is the most common type, usually affects women of childbearing age. It spreads slowly and is the least dangerous type of thyroid cancer.

The treatment of thyroid cancer depends on the type of thyroid cancer. Surgery is most often performed where the entire thyroid gland is usually removed. If the doctor suspects that the cancer has spread to lymph nodes in the neck, these will also be removed during surgery. Radiation therapy may be performed using external beam (x-ray) radiation or by taking radioactive iodine by mouth. Radiation therapy may be performed with or without surgery. After treatment, thyroid hormone needs to be taken to replace hormones that the glands previously made. The dose administered is usually a little higher than what is normally needed and this appears to help in the recurrence of the cancer. If the cancer does not respond to surgery or radiation and has spread to other parts of the body, chemotherapy may be used, but this is only effective for a small number of patients.

Accurate diagnosis of the cancer type is highly important in the management of the disease, since various forms can have significantly different prognosis. Currently, the diagnosis of thyroid cancers is based on fine needle aspiration cytology, which is known to foe subject to a high degree of inter-observer variability. The development of molecular diagnostic tools that can accurately classify the various types of thyroid carcinomas, either as stand-alone markers, or in conjunction with other markers and cytology, are of paramount importance.

miRNAs are small, endogenous, non-coding RNAs that are negative regulators of many cellular processes, some of which have been implicated in carcinogenesis. They have emerged as a new class of biomarkers that can classify tumors with greater accuracy than other molecular markers. As opposed to mRNA, miRNAs are stable in archived biological specimens and can be easily detected and analyzed with existing technologies. Therefore, they appear to be excellent candidates for retrospective studies based on archived biological specimens.

To date, few studies have investigated miRNAs in thyroid cancers. Although some studies have identified promising miRNA markers that could distinguish between several thyroid cancer types, these studies have not investigated the full profile of human miRNAs, and have generally had difficulty assessing miRNA expression due to the small amount of tissue obtained from fine needle aspirate.

### SUMMARY

In one aspect, an exemplary embodiment provides methods of screening for thyroid cancer or a potential for developing thyroid cancer in an individual subject in need thereof, where the methods comprise: (a) determining expression levels of at least one miRNA selected from a first group of specific miRNAs; (b) comparing the expression levels of the at least one miRNA from the first group with the expression levels of the corresponding miRNA established for a control individual not having thyroid cancer, and (c) identifying differences in the miRNA expression levels between the at least one sample from said individual subject and miRNA expression levels established for a control individual not having thyroid cancer. Exemplary methods can also comprise the steps of: (d) determining expression levels of at least one miRNA selected from a second group of specific miRNAs. (e) comparing the expression levels of the at least one miRNA from the second group with expression levels of the corresponding miRNA established for a control individual not having thyroid cancer; and (f) identifying differences in the miRNA expression levels for the at least one sample from said individual and miRNA expression levels established for a control individual not having thyroid cancer.

In another aspect, exemplary embodiment provide methods of diagnosing thyroid cancer, or a potential for developing thyroid cancer in an individual subject in need thereof, where the method comprises: a) determining expression levels of at least one miRNA selected from a first group of specific miRNAs; b) comparing the expression levels of the at least one miRNA from the first group and expression levels of at least one miRNA from a second group with expression levels of the corresponding miRNAs established for a control individual not having thyroid cancer, and c) identifying differences in the expression levels of the miRNAs between the individual and expression levels established for a control individual not having thyroid cancer. In an exemplary embodiment, the method further includes correlating the expression levels of the miRNAs with a diagnosis of thyroid cancer and/or a potential to develop thyroid cancer.

In another aspect, exemplary embodiment provide methods of treating an individual having thyroid cancer, where the methods comprise: a) determining expression levels of at least one miRNA selected from a first group of specific miRNAs; b) comparing the expression levels of the at least one miRNA from the first group and expression levels of at least one miRNA from a second group with expression levels of the corresponding miRNAs established for a control individual not having thyroid cancer; c) identifying differences in the expression levels of the miRNAs between the individual subject and the expression levels established for a control individual not having thyroid cancer; d) correlating the expression levels with at least one of a diagnosis of thyroid cancer and/or a potential to develop thyroid cancer; and e) treating the individual subject, based on the diagnosis, with at least one of an effective amount of radiation and a chemotherapeutic agent.

The applicability of the present teachings to other areas will become apparent from the detailed description provided hereinafter. It should be understood that the detailed description and specific examples, while indicating certain embodiments of the present teachings, are intended for purposes of illustration only and are not intended to limit the scope of the invention.

### BRIEF DESCRIPTION OF THE DRAWINGS

FIG. 1 shows a heat map of normal tissue versus papillary carcinomas (PC) with t-test results having p<0.01.

FIG. 2 shows a heat map of normal tissue versus follicular carcinomas (FC) with t-test results having p<0.01.

FIG. 3 shows a heat map of normal tissue versus follicular adenomas (FA) with t-test results having p<0.01.

FIG. 4 shows a heat map of normal tissue versus nodular hyperplasias (NH) With t-test results having p<0.01.

FIG. 5 shows a heat map of papillary carcinomas (PC) versus follicular carcinomas (FC) with t-test results having p<0.05.

FIG. 6 shows a heat map of papillary carcinomas (PC) versus follicular adenomas (FA) with t-test results having p<0.05.

FIG. 7 shows a heat map of papillary carcinomas (PC) versus nodular hyperplasias (NH) with t-test results having p<0.05.

FIG. 8 shows a heat map of nodular hyperplasias (NH) versus follicular adenomas (FA) with t-test results having p<0.05.

FIG. 9 shows a heat map of nodular hyperplasias (NH) versus Follicular Carcinomas (FC) with t-test results having p<0.05.

FIG. 10 shows a heat map of follicular adenomas (FA) versus Follicular Carcinomas (FC) with t-test results having p<0.05.

FIG. 11 shows a heat map of papillary carcinomas (PC) versus normal tissue with t-test results having p<0.05.

FIG. 12 shows a heat map of follicular adenomas (FA) versus normal tissue with t-test results having p<0.01 or 0.055.

FIG. 13 shows a heat map of follicular carcinomas (FC) versus normal tissue with t-test results having p<0.01 or 0.05.

FIG. 14 shows a heat map of nodular hyperplasias (NH) versus normal tissue with t-test results having p<0.01 or 0.05.

FIG. 15 shows a heat map of 4 Types of Thyroid Cancer versus normal tissue with t-test results having p<0.01.

FIG. 16 shows a heat map of nodular hyperplasias (NH) versus follicular carcinomas (FC) with t-test results having p<0.05.

FIG. 17 shows a heat map of nodular hyperplasias (NH) versus follicular adenomas (FA) with t-test results having p<0.05.

FIG. 18 shows a heat map of nodular hyperplasias (NH) versus follicular carcinomas (FC) with t-test results having p<0.05.

FIG. 19 shows a heat map of follicular carcinomas (FC) versus follicular adenomas (FA) with t-test results having p<0.05.

FIG. 20 shows a heat map of nodular hyperplasias (NH) versus follicular carcinomas (FC) with t-test results having p<0.05.

FIG. 21 shows a heat map of a comparison of miRNAs from follicular carcinomas (FC), nodular hyperplasias (NH), follicular adenomas (FA) and papillary carcinomas (PC) with t-test results having p<0.05.

FIG. 22 shows a heat map of “early” versus “advanced” thyroid cancers with t-test results having p<0.01, where nodular hyperplasias (NH) and follicular carcinomas (FC) are termed “early” and follicular adenomas (FA) and papillary carcinomas (PC) are termed “advanced”.

FIG. 23 shows a Venn diagram of the overlap of the number of miRNAs identified from follicular carcinoma (FC) and papillary carcinoma (PC) samples with t-test results having p<0.01.

FIG. 24 shows a Venn diagram of the overlap of the number of miRNAs identified from follicular adenoma (FA) and follicular carcinoma (FC) samples with t-test results having p<0.01.

FIG. 25 shows a Venn diagram of the overlap of the number of miRNAs identified from follicular carcinoma (FC) and nodular hyperplasia (NH) samples with t-test results having p<0.01.

FIG. 26 shows a Venn diagram of the overlap of the number of miRNAs identified from follicular adenomas (FA), follicular carcinoma (FC) and papillary carcinoma (PC) samples with t-test results having p<0.01.

FIG. 27 shows a Venn diagram of the overlap of the number of miRNAs identified from follicular adenomas (FA) and papillary carcinoma (PC) samples with t-test results having p<0.01.

FIG. 28 shows a Venn diagram of the overlap of the number of miRNAs identified from papillary carcinoma (PC) and nodular hyperplasia (NH) samples with t-test results having p<0.01.

FIG. 29 shows a Venn diagram of the overlap of the number of miRNAs identified from papillary carcinoma (PC) and nodular hyperplasia (NH) samples with t-test results having p<0.01.

FIG. 30 shows a Venn diagram of the overlap of the number of miRNAs identified from follicular adenomas (FA), follicular carcinomas (FC), papillary carcinoma (PC) and nodular hyperplasia (NH) samples with t-test results having p<0.01.

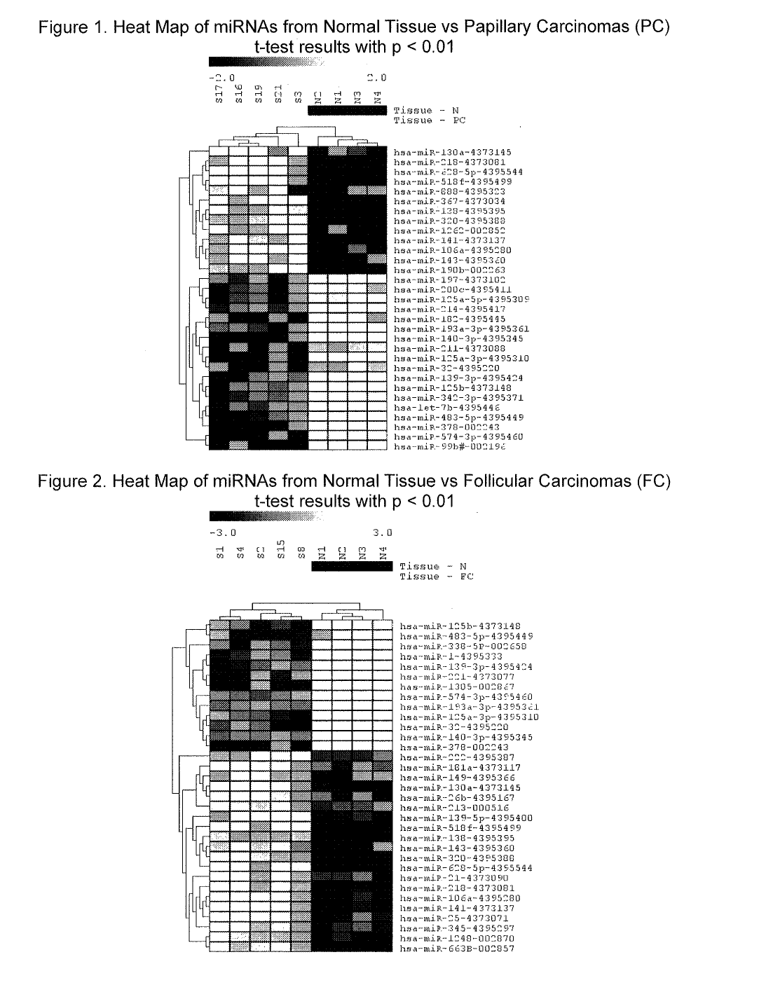

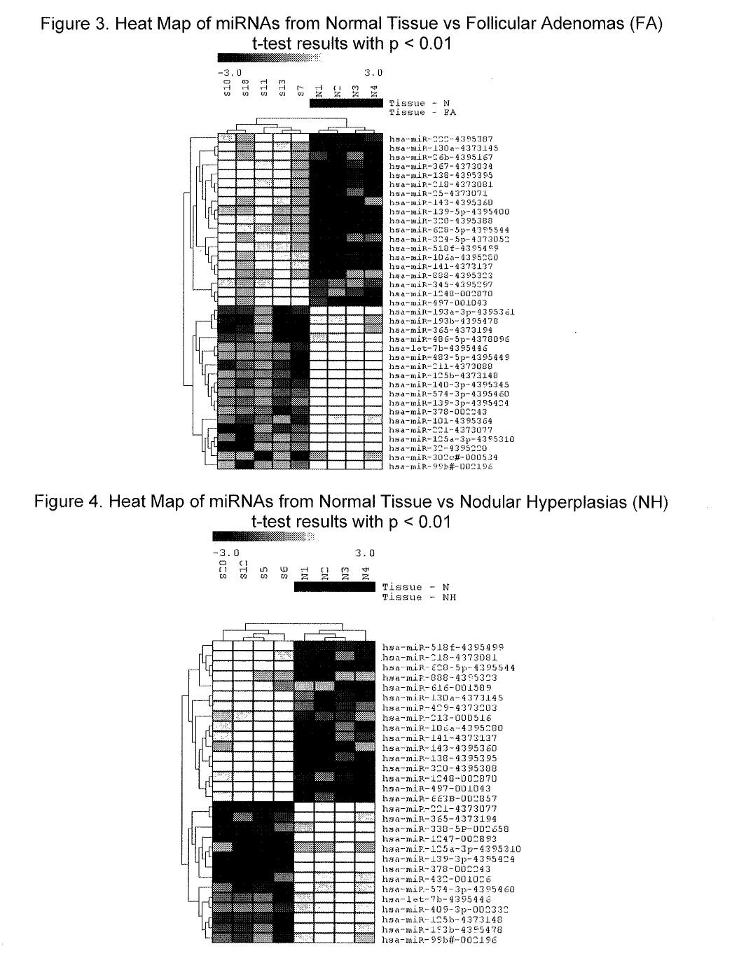

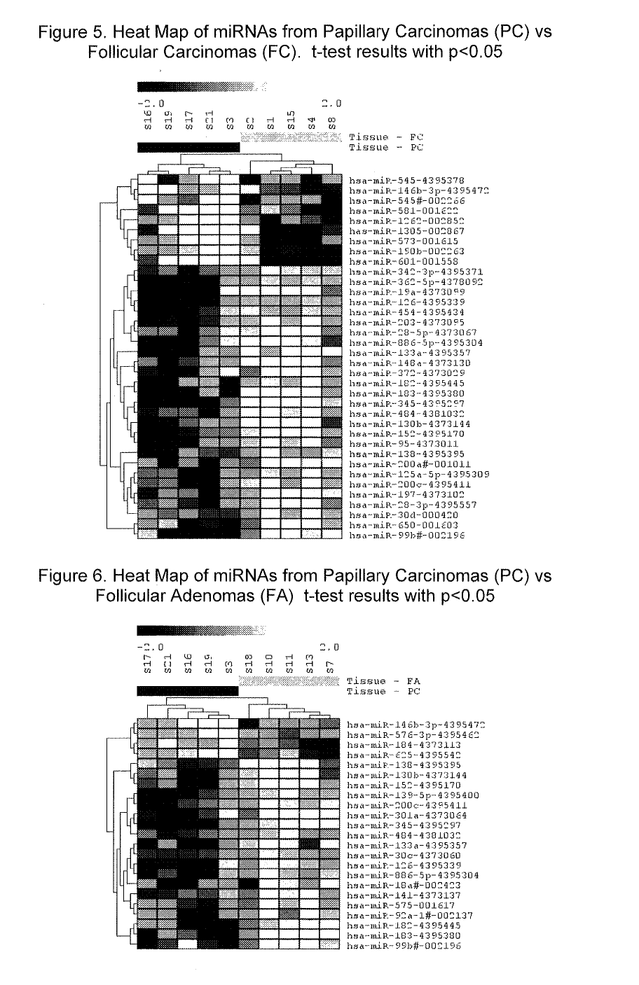

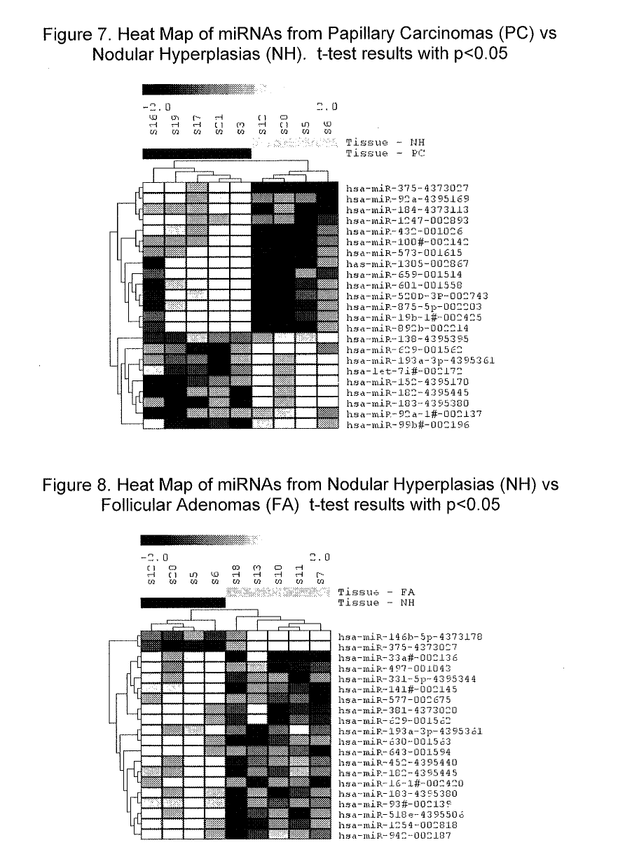

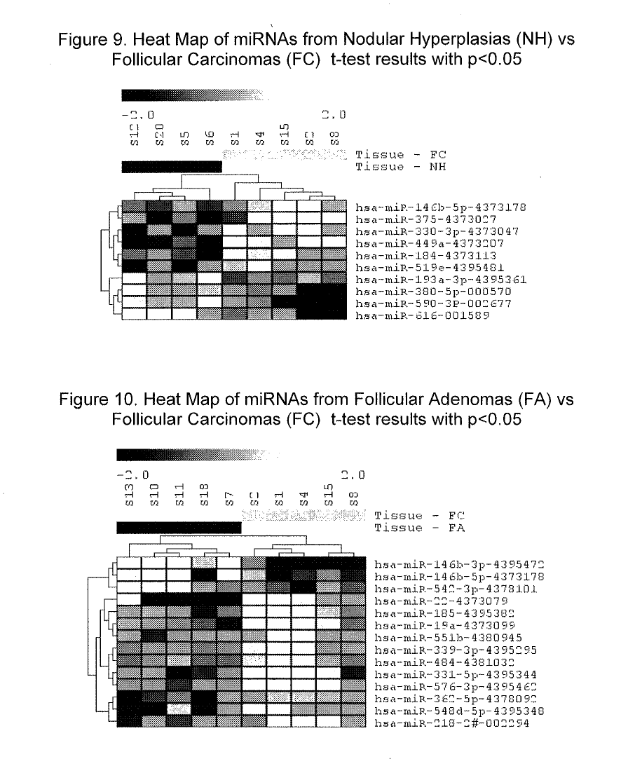

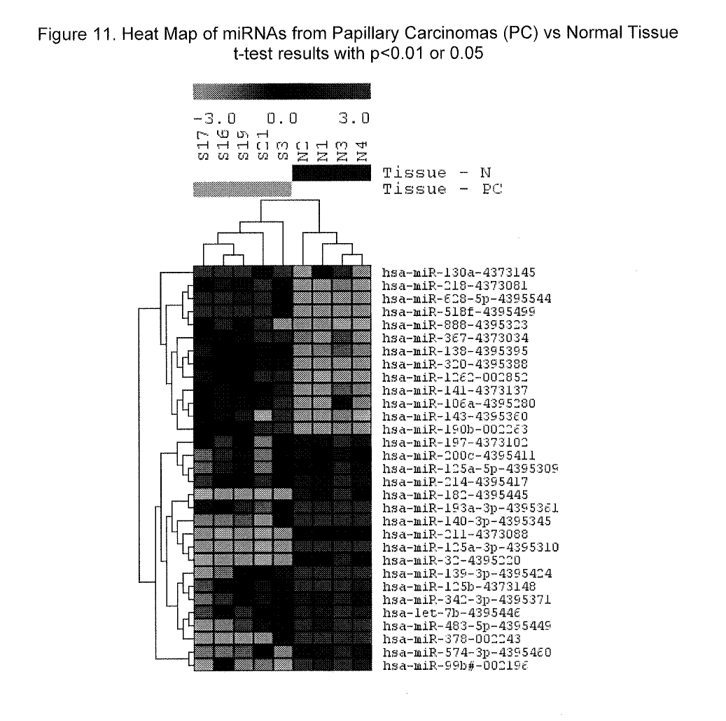

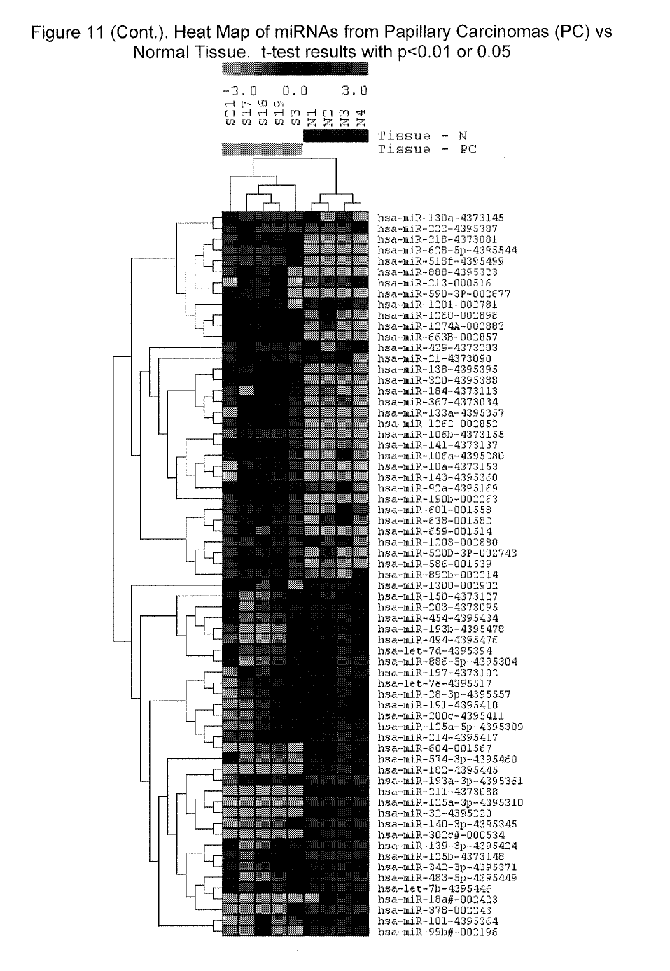

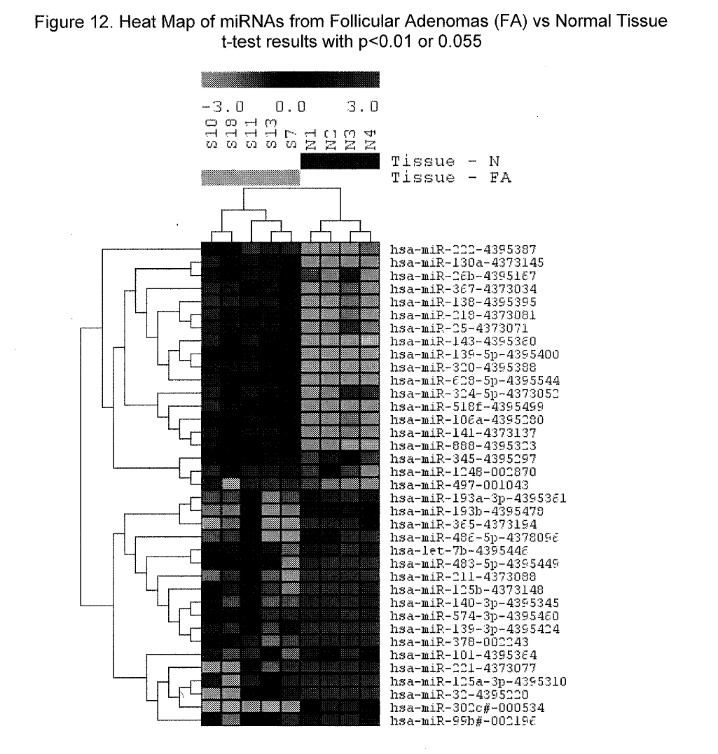

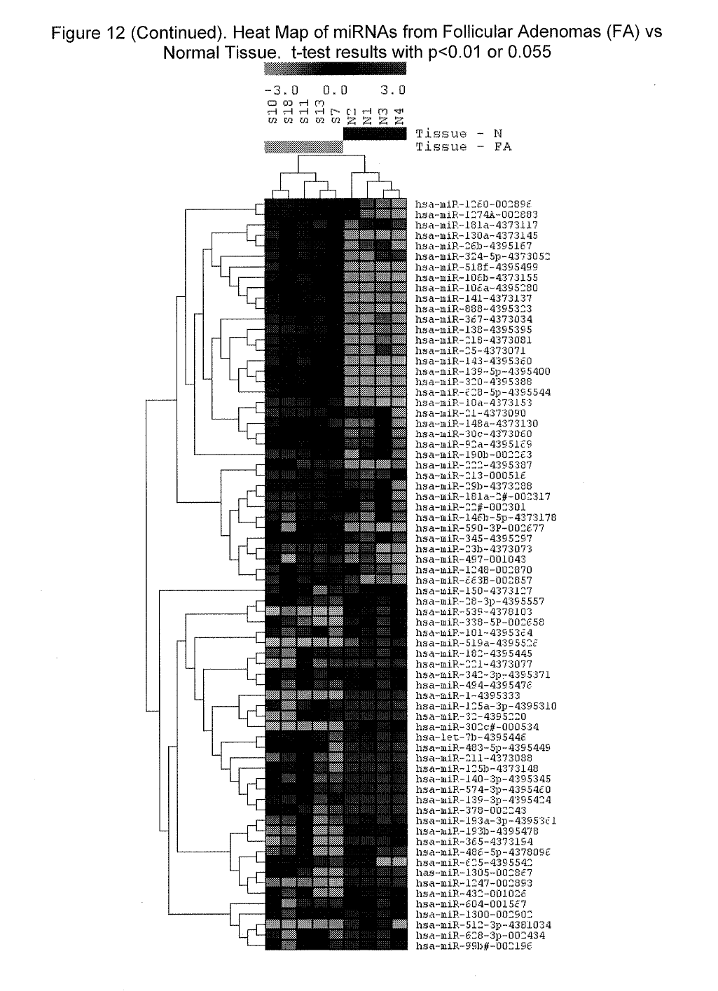

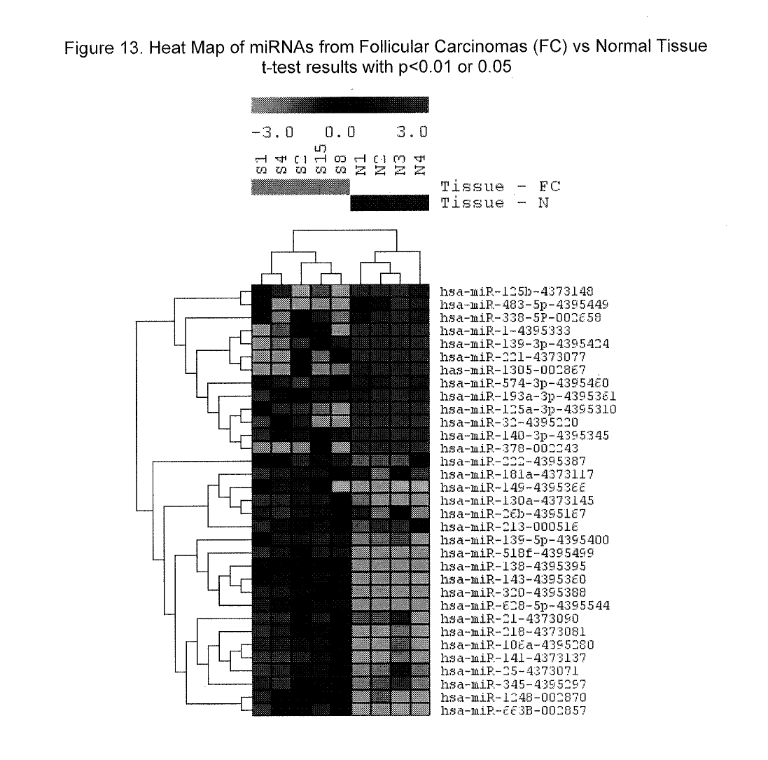

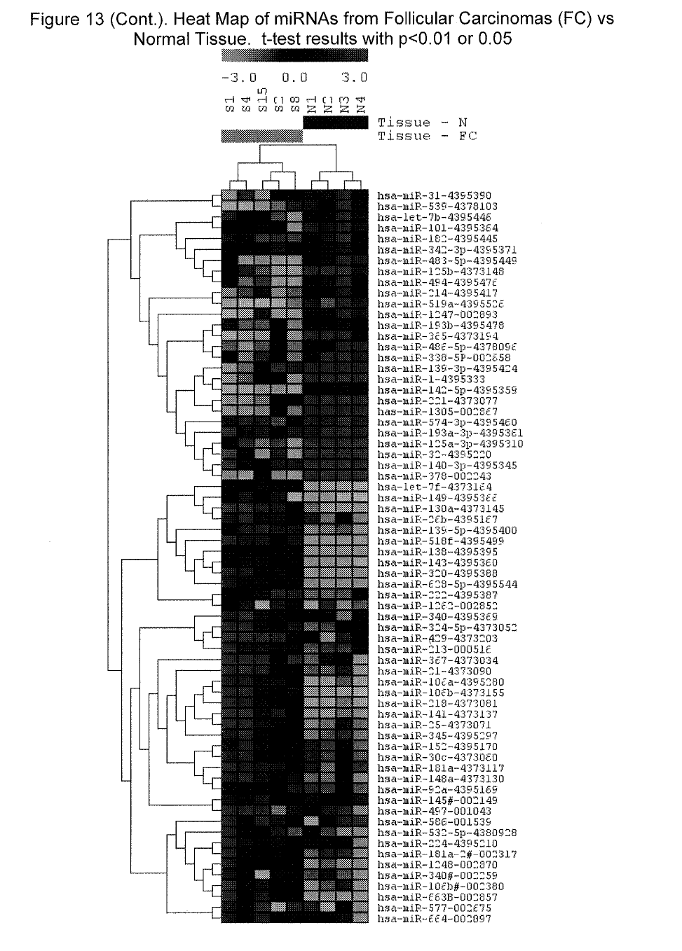

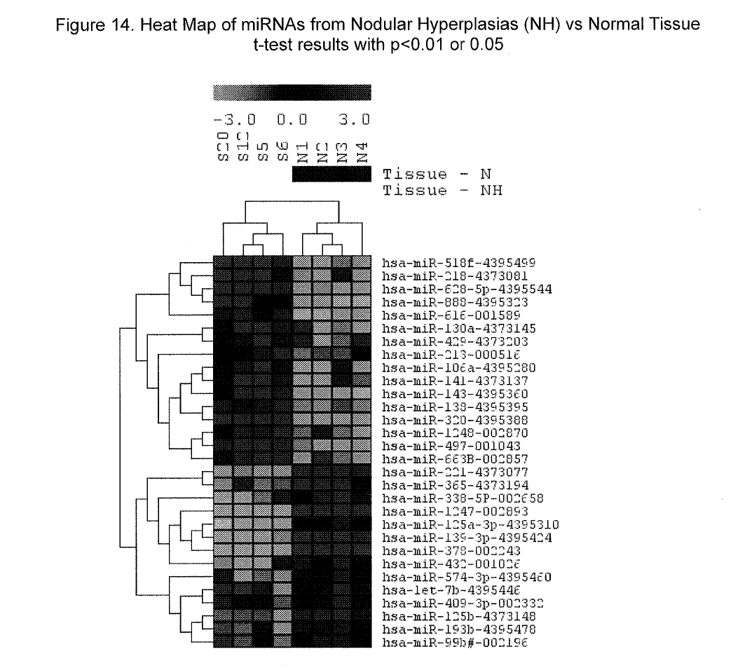

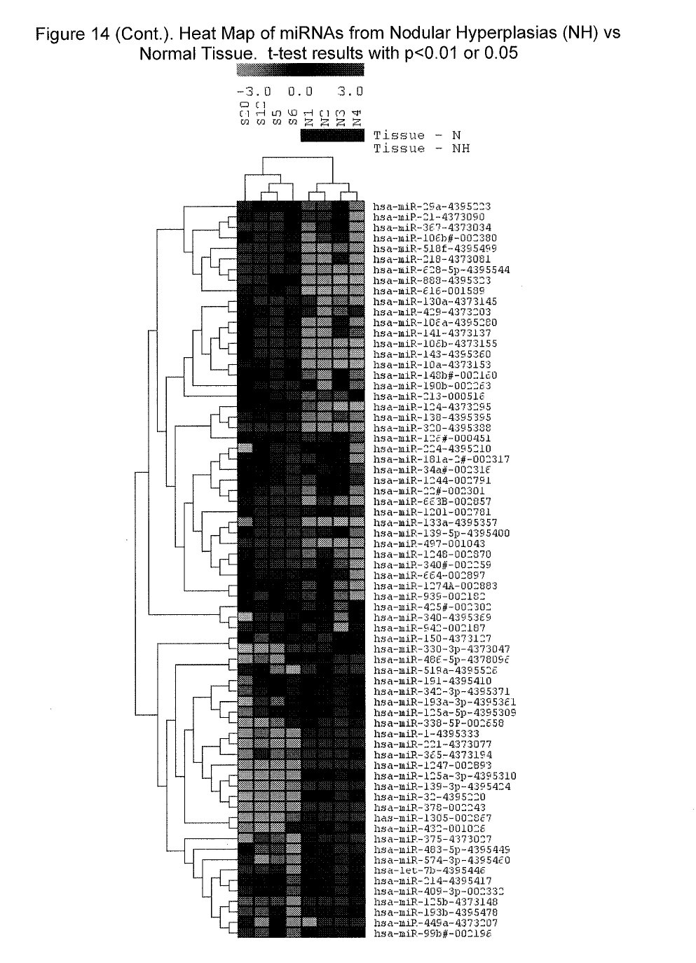

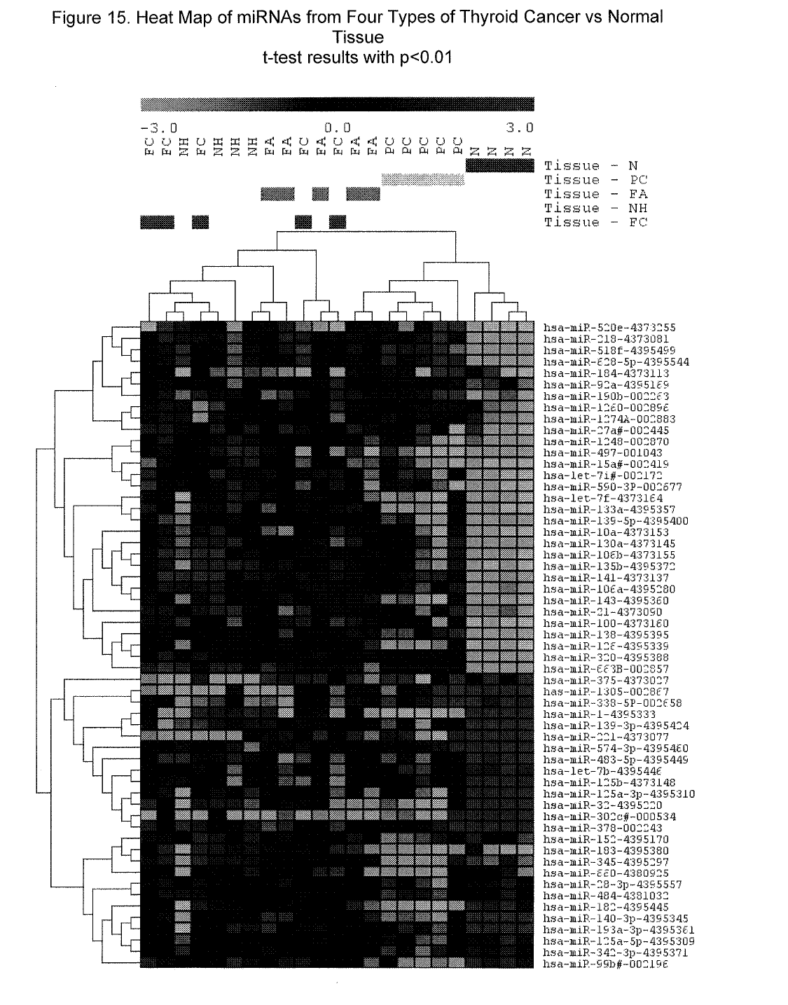

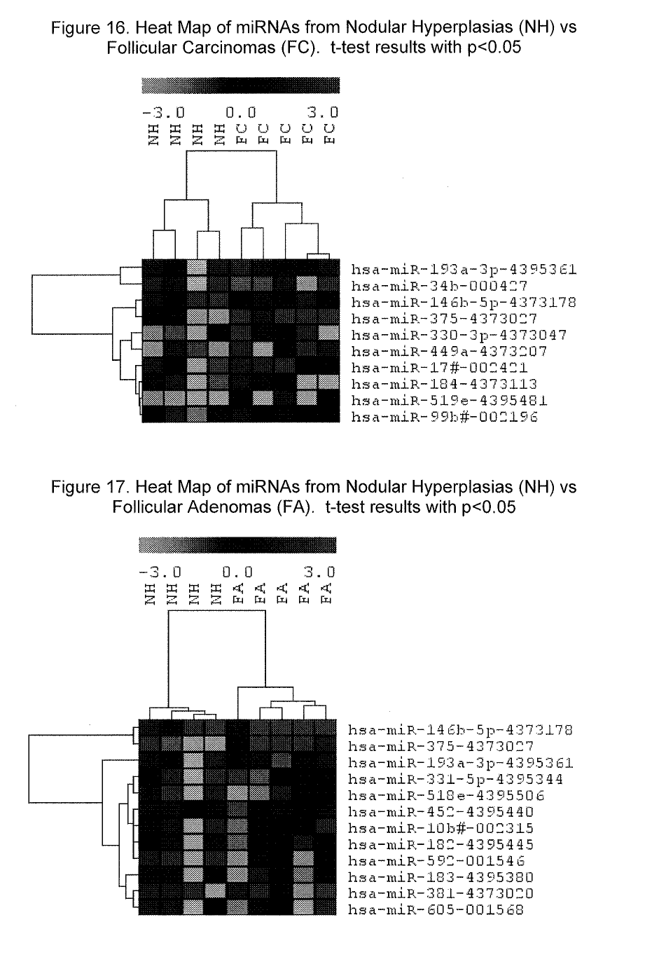

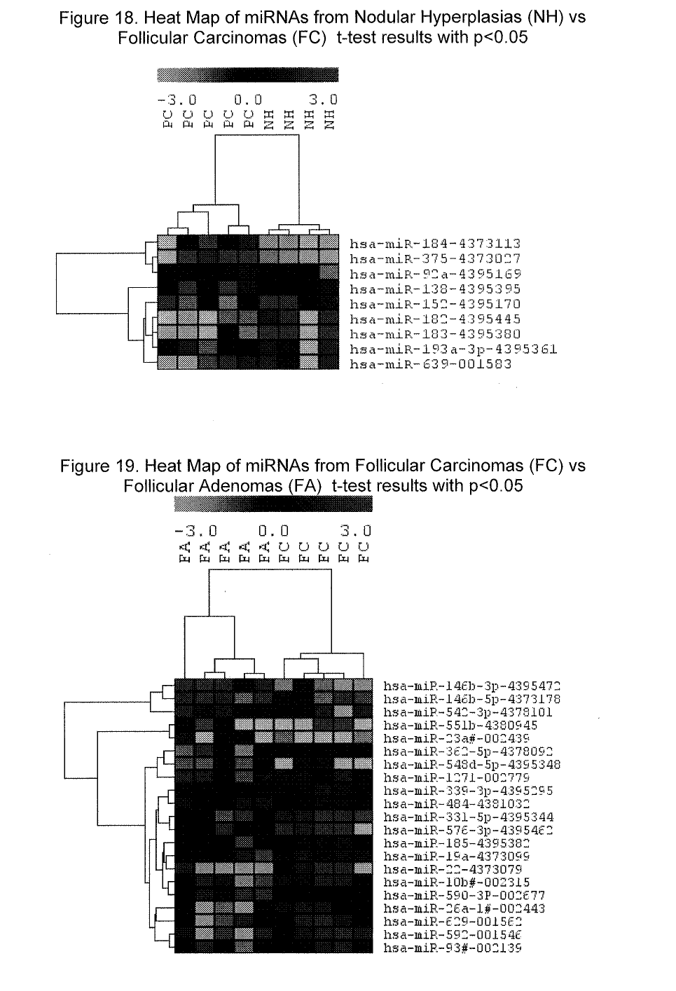

### DETAILED DESCRIPTION

Micro RNAs (miRNAs) are naturally-occurring 19 to 25 nucleotide transcripts found in over one hundred distinct organisms, including fruit flies, nematodes and humans. MicroRNAs are small, noncoding RNAs that influence gene regulatory networks by post-transcriptionaI regulation of specific messenger RNA (mRNA) targets via specific base-pairing interactions. More than 9,500 miRNAs have been identified and are recorded on a miRNA registry maintained by the Sanger Institute and available on its website microRNA@sanger.ac.uk. New miRNA sequences continue to be discovered by sequencing small RNA (e.g., 18-25 nucleotides in length) isolated from normal or diseased cells; quite possibly the number of miRNA sequences may be on par with the number of coding mRNA in a mammalian cell.

The miRNAs are typically processed from 60- to 70-nucleotide foldback RNA precursor structures, which are transcribed from the miRNA gene. The miRNA precursor processing reaction requires Dicer RNase III and Argonaute family members (Sasaki et al. (2003), Genomics 82, 323-330). The miRNA precursor or processed miRNA products are easily detected, and an alteration in the levels of these molecules within a cell can indicate a perturbation in the chromosomal region containing the miRNA gene.

Over-expression and silencing of specific miRNAs have been described in a number of diseases, including cancer. The miR-17-92 cluster is overexpressed in tumor samples from lymphoma patients, and this overexpression is correlated with amplification of the particular region of chromosome 13 in which the miRNA cluster is located (He et al., 2005, Nature 435:828-33); miR-342 is commonly suppressed in human colorectal cancer (Grady et al., 2008, Oncogene, Feb. 11, 2008, No: 18264139); miR-15 and miR-16 are under-expressed in chronic lymphocytic leukemia as a result of a deletion on chromosome 13 (Calin et al., 2005 Proc. Natl. Acad. Sci. USA 99:15524-29); reduced expression of let-7 miRNA is correlated with poor prognosis in lung cancers (Takamizawa et al., 2004, Cancer Res. 64:3753-56); and let-7 may act as a tumor suppressor by inhibiting the expression of the RAS oncogene lung tissue (Johnson et al., 2005, Cell 120:635-47).

MiRNAs are also involved in the development and function of the cardiovascular system. For example, specific miRNAs have been implicated in vascular angiogenesis and cardiomyocyte apoptosis, and also in the development of cardiac hypertrophy, arrhythmia, and heart failure, and in numerous other diseases and developmental processes, including schizophrenia, Alzheimer's disease, immune cell development and pluripotency, nervous system development, endocrine disease, including diabetes, development of the pancreas, Fragile X Syndrome, cutaneous wound healing, cell cycle progression, transplanted tissue rejection, hypoxia, and skeletal muscle differentiation. MiRNAs are also expressed by viruses, and target genes of those miRNAs have been identified.

Given the important functional role of miRNA in disease, this set of nucleic acid molecules contains candidates for diagnosing and prognosing disease, and monitoring response to therapies in a wide variety of patients and in subjects prior to manifesting disease. This potential utility is severely limited however by current methods, which are limited to extracting RNA from cells. Further, the tissue responsible for many disease conditions is not accessible to biopsy or may not be detectable for a late stage of disease. There remains an unmet need to diagnose disease using miRNA in a readily available biological sample, such as, for example, blood, serum, plasma, combinations thereof, and the like. miRNA dysregulation contributes to cancer diagnosis and therapy and the identification of specific diagnostics and therapeutic targets is needed to provide new and useful tools for improved management of disease.

Cancers are a significant source of mortality and morbidity in the U.S. and throughout the world. However, cancers are a large and varied class of diseases with diverse etiologies. Researchers therefore have been unable to develop treatments or diagnostic tests which cover more than a few types of cancer. Accordingly, there is a need fur a reliable indicator of an individual predicted disease course to help clinicians to identify these patients that will respond to treatment, those patients that will progress to a more advanced state of the disease and those patients with emerging resistance to treatment.

### Definitions:

The following definitions are provided for specific terms which are used in the following written description.

As used herein, the singular form “a”, “an” and “the” include plural references and can mean “at least one” unless the context clearly dictates otherwise.

The term thyroid cancer includes follicular carcinomas (FC), follicular adenomas (FA), papillary carcinomas (PC), and nodular hyperplasia (NH). Although nodular hyperplasia is a benign lesion, it is included within this term for purposes of this application.

As used herein, “microRNA” or “miRNA” means a small, noncoding RNA sequence of 5 to 40 nucleotides in length that can be detected in a biological specimen. MicroRNAs (miRNAs) are short ribonucleic acid (RNA) molecules, on average only 22 nucleotides long. miRNAs are post-transcriptional regulators that bind to complementary sequences on target messenger RNA transcripts (mRNAs), usually resulting in translational repression and gene silencing.

Exemplary embodiments provide methods of screening for thyroid cancer or a potential for developing thyroid cancer in an individual subject in need thereof, where the methods comprise: (a) determining expression levels of at least one miRNA selected from a first group of specific miRNAs in at least one sample from the individual subject; (b) comparing the expression levels of the at least one miRNA from the first group with expression levels of the corresponding miRNA established for a control individual not having thyroid cancer, and (c) identifying differences in the miRNA expression levels between the at least one sample from said individual subject and miRNA expression levels established for a control individual not having thyroid cancer.

In an embodiment, a method of screening an individual subject in need for thyroid cancer or a potential for developing thyroid cancer in an individual subject in need thereof comprises:
(a) determining expression levels of at least one miRNA selected from a first group consisting of miR-1 miR-7b, miR-26b, miR-101, miR-106a, miR-130a, miR-139, miR-141, miR-143, miR-149, miR-182, miR-190b, miR-193a, miR-193b, miR-197, miR-200c, miR-211, miR-214, miR-218, miR-302c, miR-320, miR-324, miR-338, miR-342, miR-365, miR-367, miR-378, miR-409, miR-429, miR-432, miR-483, miR-486, miR-497, miR-518f, miR-574, miR-574, miR-616, miR-628, miR-663b, miR-888, miR-1247, miR-1248, miR-1248, miR-1262, and miR-1305 in at least one sample from said individual subject;
(b) comparing the expression levels of the at least one miRNA from the first group with expression levels of the corresponding miRNA established for a control individual not having thyroid cancer, and
(c) identifying differences in the miRNA expression levels between the at least one sample from said individual subject and the miRNA expression levels established for a control individual not having thyroid cancer.
   Exemplary methods can also comprise the steps of:
(d) determining expression levels of at least one miRNA selected from a second group consisting of: miR-21, miR-25, miR-32, miR-99b*, miR-125a, miR-125b, miR-136, miR-140, miR-181a, miR-213, miR-221, miR-222, and miR-345;
(e) comparing the expression levels of the at least one miRNA from the second group with expression levels of the corresponding miRNA established for a control individual not having thyroid cancer; and
(f) identifying differences in the miRNA expression levels at least one sample from said individual subject and miRNA expression levels established for a control individual not having thyroid cancer.

In another embodiment, when the expression levels for the at least one miRNA from the first group and the expression levels for the at least one miRNA from the second group indicate an over-expression compared to the levels established for a control individual not having thyroid cancer, the method further comprises correlating the expression levels with a diagnosis of a least one of thyroid cancer and a potential for developing thyroid cancer and optionally treating said individual subject, in accordance with the diagnosis, by at least one of surgical removal of thyroid tissue from the individual subject and administering an effective amount of radiation and/or a chemotherapeutic agent, such as doxorubicin or cisplatin, to said individual subject.

In a further embodiment, when the expression levels for the at least one miRNA from the first group and the expression levels for the at least one miRNA from the second group indicates an under-expression compared to the levels established for a control individual not having thyroid cancer, the method further comprises correlating the expression levels with a diagnosis of at least one of thyroid cancer and a potential for developing thyroid cancer and optionally treating said individual subject, in accordance with the diagnosis, by at least one of surgical removal of thyroid tissue from the individual subject and administering an effective amount of radiation and/or a chemotherapeutic agent to the individual subject.

In yet another embodiment, when the expression levels for the at least one miRNA from the first group or from the second group indicate an over-expression compared to the levels established for a control individual not having thyroid cancer and the expression levels for the at least one miRNA from the first group or from the second group indicate an under-expression compared to the levels established for the same miRNAs established for a control individual not having thyroid cancer the method further comprises correlating the expression levels with a diagnosis of at least one of thyroid cancer and a potential for developing thyroid cancer and optionally treating said individual subject, in accordance with the diagnosis, by at least one of surgical removal of thyroid tissue from the individual subject and administering an effective amount of radiation and/or a chemotherapeutic agent to the individual subject.

In an embodiment, the thyroid cancer is a follicular adenoma, a follicular carcinoma, a papillary carcinoma. In a further embodiment, a total of at least 5, 10, 15, 20, 25, 30, 35 or 40 miRNAs from the first group are present at levels indicating over-expression or under-expression when compared to expression levels for the same miRNAs established for a control individual not having thyroid cancer. In another embodiment, a total of at least 5 miRNAs from the first and second groups have expression levels indicating over-expression or under-expression when compared to expression levels for the same miRNAs established for a control individual not having thyroid cancer. In yet another embodiment, a total of at least 5, 10, 15, 20, 25, 30, 35 or 40 miRNAs from the first group and the second group have expression levels indicating over-expression or under-expression when compared to expression levels for the same miRNAs established for a control individual not having thyroid cancer. In a further embodiment, when the expression levels for the at least one miRNA from the first group or from the second group indicate an over-expression compared to the levels established for a control individual not having thyroid cancer and the expression levels for the at least one miRNA from the first group or from the second group indicate an under-expression compared to the levels established for the same miRNAs established for a control individual not having thyroid cancer the method further comprises correlating the expression levels with a diagnosis of at least one of thyroid cancer and a potential for developing thyroid cancer and optionally treating said individual subject, in accordance with the diagnosis, by at least one of surgical removal of thyroid tissue from the individual subject and administering an effective amount of radiation and/or a chemotherapeutic agent to the individual subject.

In another aspect, exemplary embodiments provide methods of diagnosing thyroid cancer, or a potential for developing thyroid cancer, in an individual subject in need thereof. In an embodiment, the method of diagnosing thyroid cancer, or a potential for developing thyroid cancer, in an individual subject in need thereof comprises:
a) determining expression levels of at least one miRNA selected from a first group consisting of miR-1, miR-7b, miR-26b, miR-101, miR-106a, miR-130a, miR-139, miR-141, miR-143, miR-149, miR-182, miR-190b, miR-193a, miR-193b, miR-197, miR-200c, miR-211, miR-214, miR-218, miR-302c*, miR-320, miR-324, miR-338, miR-342, miR-365, miR-367, miR-378, miR-409, miR-429, miR-432, miR-483, miR-486, miR-497, miR-518f, miR-574, miR-574, miR-616, miR-628, miR-663b, miR-888, miR-1247, miR-1248, miR-1248, miR-1248, miR-1262, and miR-1305, and at feast one miRNA from a second group consisting of miR-21, miR-25, miR-32, miR-99b*, miR-125a, miR-125b, miR-138, miR-140, miR-181a, miR-213, miR-221, miR-222, and miR-345, in at least one sample from said individual subject;
b) comparing the expression levels of the at least one miRNA from the first group and expression levels of at least one miRNA from a second group with expression levels of the corresponding miRNAs established for a control individual not having thyroid cancer, and
c) identifying differences in the expression levels of the miRNAs between the individual subject and expression levels established for a control individual not having thyroid cancer.
   In exemplary embodiments, the method includes correlating the expression levels with a diagnosis of thyroid cancer and/or a potential for developing thyroid cancer.

In another aspect, exemplary embodiments provide methods of treating an individual having thyroid cancer and/or a potential for developing thyroid cancer. In an embodiment the method of treating an individual subject having thyroid cancer comprises:
a) determining expression levels of at least one miRNA selected from a first group consisting of miR-1, miR-7b, miR-26b, miR-101, miR-1.06a, miR-130a, miR-139, miR-141, miR-143, miR-149, miR-182, miR-190b, miR-193a, miR-193b, miR-197, miR*200c, miR-211, miR-214, miR-218, miR-302c*, miR-320, miR-324, miR-338, miR-342, miR-365, miR-367, miR-378, miR-409, miR-429, miR-432, miR-483, miR-486, miR-497, miR-518f, miR 574, miR-574, miR-616, miR-628, miR-663b, miR-888, miR-1247, miR-1248, miR-1248, miR-1248, miR-1262, and miR-1305, and at least one miRNA from a second group consisting of miR-21, miR-25, miR-32, miR-99b*, miR-125a, miR-125b, miR-138, miR-140, miR-181a, miR-213, miR-221, miR-222, and miR-345, in at least one sample from said individual subject:
b) comparing the levels of the at least one miRNA from the first group and the expression levels of at least one miRNA from a second group with the expression levels of the corresponding miRNAs established for a control individual not having thyroid cancer;
c) identifying differences in the expression levels of the miRNAs between the individual subject and the expression levels established for a control individual not having thyroid cancer;
d) correlating and/or comparing the expression levels with a diagnosis of thyroid cancer and/or a potential to develop thyroid cancer; and
e) treating the individual subject, based on the diagnosis, by at least one of surgically removing thyroid tissue from the individual subject and administering at least one of an effective amount of radiation and a chemotherapeutic agent to the individual subject.

In another embodiment, the method further comprises the step of determining the expression levels of at least one miRNA from the first group and at least one miRNA from the second group from at least one sample obtained from said individual subject after step e). In another embodiment, the method further comprises the step of comparing the expression levels of the at least one miRNA from a first group and at least one miRNA from a second group with the expression levels of the corresponding miRNAs between at least one sample before treating the individual subject with radiation and/or a chemotherapeutic agent or removing thyroid tissue. In yet another embodiment, the method further comprises g) establishing a treatment plan based on the comparison of the expression levels of the at least one miRNA from a first group and at least one miRNA from a second group with the expression levels of the corresponding miRNAs established for a control individual not having thyroid cancer before treating the individual subject with radiation and/or a chemotherapeutic agent or removing thyroid tissue.

Another aspect of the invention is microarrays comprising the specific groups of the above-described miRNAs. Microarrays can be used to measure the expression levels of large numbers of miRNAs simultaneously. Microarrays can be fabricated using a variety of technologies, including printing with fine-pointed pins onto glass slides, photolithography using pre-made masks, photolithography using dynamic micromirror devices, ink-jet printing, or electrochemistry on microelectrode arrays. Also useful, are microfluidic TaqMan Low-Density Arrays based on an army of microfluidic qRT-PCR reactions as well as related microfluidic qRT-PCR based methods.

Exemplary embodiments provide a method of manufacturing a report, the method comprising (a) contacting a biological sample, preferably a sample of blood, or material derived from the blood, with means of detecting miRNA; measuring expression levels of at least one miRNA of the first group and optionally from the second group, as described above; and transforming by a computing means said expression level measurements into a level of at least one said miRNA in the biological sample; and producing a report describing said levels of said miRNA in a tangible medium.

A major benefit of the report is to provide a means of diagnosing or measuring the presence of a disease in a clinical setting, to thereby accelerate treatment of the disease to an earlier stage than afforded by conventional diagnostic means. Ultimately, a report will provide improved health care for the patient, increased certainty for the health care provider's diagnosis, decreased cost of health care for both health care managers and insurance providers, and improved public health. This, and other exemplary methods disclosed herein, may also allow a physician to provide targeted treatment for the stage or type of thyroid cancer or potential for thyroid cancer.

Any individual having an interest in measuring the presence of a disease in a subject can cause a report to be manufactured, including a subject or patient, a medical doctor, a physician, a health management organization (HMO), a clinic, a health care provider, a health insurer, a company involved in reimbursing an insurance claim or in negotiating the cost of a diagnostic service. A pharmaceutical company can cause a report to be manufactured during a clinical trial to measure the extent of efficacy of an experimental therapy. In a health care setting, the cost of the report can be paid by an insurance provider who causes the report to conform to certain specifications which are required for payment. In the same way, the doctor or other health care provider can cause the report to contain information relevant to a diagnosis or prognosis.

The report must contain information about the expression levels of at least one of the miRNAs in the biological sample. The expression levels of the miRNA can be described with respect to an absolute concentration or amount, or can be relative to normal levels in the population or relative to prior measurements in the same subject. The report can optionally include recommended treatment or a treatment modality for the cancer or pre-cancer condition.

The report can instead, or in addition, include a probability assessment that evaluates the relative risk of a cancer, or the extent of an existing cancer. The report can provide information to the requestor that enables a diagnosis or prognosis related to a cancer. The manufactured report can also contain a diagnosis or prognosis resulting from analysis performed by a diagnostic service provider utilizing information about the levels of at least one of the protein markers in the blood.

The report can be produced in writing. The writing can be in a printed form, or be provided through an electronic means. If electronic, it can be provided through an email account, a secure web sight or an external FTP site.

Diagnostic Kits

Diagnostic kits adapted for the determination of the expression levels of miRNAs and diagnoses of disease are provided herein. Such kits can include materials and reagents adapted to specifically determine the presence and/or amount of miRNA expression or group of miRNAs' expression selected to be a useful diagnostic or biomarker of disease in a sample of body fluid. The kit can include nucleic acid molecules or probes in a form suitable for the detection of the miRNAs. The nucleic acid molecules can be in any composition suitable for the use of the nucleic acid molecules according to the instructions. The kit can include a detection component, such as a microarray, a labeling system, a cocktail of components (e.g., suspensions required for any type of PCR, especially real-time quantitative RT-PCR), membranes, color-coded beads, columns and the like. Furthermore, the kit can include a container, pack, kit or dispenser together with instructions for use.

A diagnostic kit can include, for example, forward and reverse primers designed to amplify and detect the miRNA in body fluid. Many different PCR primers can be designed and adapted as necessary to amplify one or more miRNAs that are differentially expressed in a body fluid or sample and correlate to a particular disease or disorder. In one embodiment, the primers are designed to amplify a miRNA or group of miRNAs that are differentially expressed in a body fluid of an individual having cancer or at risk of developing cancer. The diagnostic kit can also contain single stranded oligonucleotide containing universal primer sequences, polyadenylated sequences, or adaptor sequences prior and a primer complementary to said sequences. The miRNA isolated from the body fluid is ligated to the single stranded oligonucleotide containing universal primer sequence, polyadenylated sequence, or adaptor sequence prior to reverse transcription and amplified with said complementary primers. In an embodiment, the kit comprises primers that amplify one of more of miRNA from the first group and optionally one of more of miRNA from the second group, as described above.

It is to be understood that this application is not limited to particular embodiments described. It is also to be understood that the terminology used herein is for the purpose of describing particular embodiments only, and is not intended to be limiting, since the scope of the present application will be limited only by the appended claims.

Unless defined otherwise, all technical and scientific terms used herein have the same meaning as commonly understood by one of ordinary skill in the art. Although any methods and materials similar or equivalent to those described herein can also be used in the practice or testing of exemplary embodiments, specific preferred methods and materials are now described.

### EXAMPLE

The micro-RNA profile of samples identified as being nodular hyperplasia (NH), follicular carcinomas (FC), follicular adenomas (FA), papillary carcinomas (PC), and normal thyroid tissue were determined and compared to each other to determine differences between the various cancers and normal tissue and to determine similarities and differences between the cancers.

### Tissue Samples

Tissue samples, each of which was over 10 years old, were obtained from paraffin-embedded blocks from different individuals from the following groups for comparison:

| Tissue Type | # Samples |
|---|---|
| Normal tissue | 4 |
| NH—nodular hyperplasia | 4 |
| FC—folicular carcinoma | 5 |
| FA—folicular adenoma | 5 |
| PC—papilary carcinoma | 5 |

### Laboratory Assays

### RNA Extraction

Total RNA (containing miRNA) is extracted from formalin fixed, paraffin embedded (FFPE) tissue slides using the RecoverAll™ Total Nucleic Acid Isolation Kit (Applied Biosystems), a kit specifically optimized for high yield recovery of miRNA from FFPE samples. In order to limit the pre-analytical variability, 20 μm thick slides were used for extraction from both normal and cancer tissues. The gross microdissection of the tissue from the slide is performed with a fine needle under a microscope using a separate HE-stained pathologically reviewed slide as a guide for microdissection. Using this procedure, the amount of surrounding stromal and normal cells to be included in the extraction is limited from sample of cancer tissue. This procedure ensured the extraction of mainly epithelial structures from samples of the normal tissue. The dissected tissue is deparafinized with xylene, treated with protease and DNase to eliminate protein and DNA contamination, ethanol precipitated, passed through the microfilter columns and RNA is recuperated by elution according to the manufacturer instructions. The quantity of the extracted RNA is then assessed using a Thermo Scientific NanoDrop™ Spectrophotometer.

miRNA Profiling

MiRNA profiling is performed using the TaqMan® Human MicroRNA Array set v3.0 (Applied Biosystems), an assay based on a quantitative real time PCR technology which makes use of the high specificity, sensitivity, and dynamic range of TaqMan® Assays. The array enables accurate quantitation of 760 human microRNAs and includes 3 TaqMan® MicroRNA Assay endogenous controls to aid in data normalization and one TaqMan® MicroRNA Assay not related to humans as a negative control. The method has been previously described in detail and this type of arrays were successfully used for miRNA profiling of FFPE tissue, serum and plasma extracted RNA, [See: Chen, C., Ridzon, D. A., Broomer, A. J., Zhou, Z., Lee, D. H., Nguyen, J. T., Barbisin, M., Xu, N. L., Mahuvakar, V. R., Anderson, M. R., Lao, K. Q., Livak, K. J., and Guegler, K. J. (2005) Real-time quantification of microRNAs by stem-loop RT-PCR. Nucleic Acids Res., 33, e179; Hoefig, K. P., Thorns, C., Roehle, A., Kaehler, C., Wesche, K. O., Repsilber, D., Branke, B., Thier, M., Feller, A. C., and Merz, H. (2008) Unlocking pathology archives for microRNA-profiling. Anticancer Res., 28, 119-123; Hui, A. B., Shi, W. Boutros, P. C., Miller, N., Pintilie, M., Fyles, T., McCready, D., Wong, D., Gerster, K., Jurisica, I., Penn, L. Z., and Liu, F. F. (2009) Robust global micro-RNA profiling with formalin-fixed paraffin-embedded breast cancer tissues. Lab Invest.; Resnick, K. E., Alder, H., Hagan, J. P., Richardson, D. L., Croce, C. M., and Cohn, D. E. (2009) The detection of differentially expressed microRNAs from the serum of ovarian cancer patients using a novel real-time PCR platform. Gynecol. Oncol., 112, 55-59; and Schmittgen, T. D., Lee, E. J., Jiang, J., Sarkar, A., Yang, L., Elton, T. S., and Chen, C. (2008) Real-time PCR quantification of precursor and mature microRNA. Methods, 44, 31-38, the disclosures of which are hereby incorporated by reference in their entirety.] Less than 500 ng of total RNA is subjected to a reverse transcription reaction using specific stem loop primers to convert all miRNAs to cDNA with the TaqMan® MicroRNA Reverse Transcription Kit (Applied Biosystems). Then, the cDNA is loaded on the array containing the specific primers and TaqMan probes for the human miRNAs and subjected to a quantitative real time PCR. Expression of the miRNAs is then calculated using the well established comparative Ct method relative to selected controls, corrected for efficiency of amplification. [See Davoren, P. A., McNeill, R. E., Lowery, A. J., Kerin, M. J., and Miller, N. (2008) Identification of suitable endogenous control genes for microRNA gene expression analysis in human breast cancer. BMC Molecular Biology 2008; 9: 76, the disclosure of which is hereby incorporated by reference in its entirety.]

miRNA expression profile for each sample is compared to that of the other tissue types to determine which specific miRNAs were over- or under-expressed in each tissue. The miRNAs that show significant over- or under-expression in one tissue type versus another, with a p-value <0.01, are arranged into heat maps and these are compared in Venn diagrams.

### Results:

10 individual miRNAs were expressed differentially between NH and FC, 12 between the NH and the FA groups, 9 between the NH and PC groups, and 21 between the FC and FA groups. Additionally, there were a greater number of miRNAs differentially expressed when the FA group was compared to the PC group and when the FC group was compared to the PC group, with 27 miRNAs and 39 miRNAs respectively.

A comparison of the levels of miRNAs expressed in samples identified as being papillary carcinomas (PC) with the levels of miRNAs expressed in samples identified as being normal tissue using a t-test with p<0.01 identified the 31 miRNAs listed in Table 1.

TABLE 1

| | |
|---|---|
| hsa-let-7s | hsa-miR-214 |
| hsa-miR-106a | hsa-miR-218 |
| hsa-miR-125a-3p | hsa-miR-32 |
| hsa-miR-125a-5p | hsa-miR-320 |
| hsa-miR-125b | hsa-miR-342-3p |
| hsa-miR-130a | hsa-miR-367 |
| hsa-miR-138 | hsa-miR-483-5p |
| hsa-miR-139-3p | hsa-miR-518f |
| hsa-miR-140-3p | hsa-msR-574-3p |
| hsa-miR-141 | hsa-miR-628-5p |
| hsa-miR-143 | hsa-miR-888 |
| hsa-miR-182 | hsa-miR-1262 |
| hsa-miR-193a-3p | hsa-miR-190b |
| hsa-miR-197 | hsa-miR-378 |
| hsa-miR-200c | hsa-miR-99b* |
| hsa-miR-211 | |

A comparison of the levels of miRNAs expressed in samples identified as being follicular carcinomas (FC) with the levels of miRNAs expressed in samples identified as being normal tissue using a t-test with p<0.01 identified the 33 miRNAs listed in Table 2.

TABLE 2

| | |
|---|---|
| hsa-miR-1 | hsa-miR-222 |
| hsa-miR-106a | hsa-miR-25 |
| hsa-miR-125a-3p | hsa-miR-26b |
| hsa-miR-125b | hsa-miR-32 |
| hsa-miR-130a | hsa-miR-320 |
| hsa-miR-138 | hsa-miR-345 |
| hsa-miR-139-3p | hsa-miR-483-5p |
| hsa-miR-139-5p | hsa-miR-518f |
| hsa-miR-140-3p | hsa-miR-574-3p |
| hsa-miR-141 | hsa-miR-628-5p |
| hsa-miR-143 | has-miR-1305 |
| hsa-miR-149 | hsa-miR-1248 |
| hsa-miR-181a | hsa-miR-213 |
| hsa-miR-193a-3p | hsa-miR-338-5P |
| hsa-miR-21 | hsa-miR-378 |
| hsa-miR-218 | hsa-miR-663B |
| hsa-miR-221 | |

A comparison of the levels of miRNAs expressed in samples identified as being follicular adenomas (FA) with the levels of miRNAs expressed in samples identified as being normal tissue using a t-test with p<0.01 identified the 37 miRNAs listed in Table 3.

TABLE 3

| | |
|---|---|
| hsa-let-7s | hsa-miR-26b |
| hsa-miR-101- | hsa-miR-32 |
| hsa-miR-106a | hsa-miR-320 |
| hsa-miR-125a-3p | hsa-miR-324-5p |
| hsa-miR-125b | hsa-miR-345 |
| hsa-miR-130a | hsa-miR-365 |
| hsa-miR-138 | hsa-miR-367 |
| hsa-miR-139-3p | hsa-miR-483-5p |
| hsa-miR-139-5p | hsa-miR-486-5p |
| hsa-miR-140-3p | hsa-miR-518f |
| hsa-miR-141 | hsa-miR-574-3p |
| hsa-miR-143 | hsa-miR-628-5p |
| hsa-miR-193a-3p | hsa-miR-888 |
| hsa-miR-193b | hsa-miR-1248 |
| hsa-miR-211 | hsa-miR-302c* |
| hsa-miR-218 | hsa-miR-378 |
| hsa-miR-221 | hsa-miR-497 |
| hsa-miR-222 | hsa-miR-99b* |
| hsa-miR-25 | |

A comparison of the levels of miRNAs expressed in samples identified as being nodular hyperplasia (NH) with the levels of miRNAs expressed in samples identified as being normal tissue using a t-test with p<0.01 identified the 30 miRNAs listed in Table 4.

TABLE 4

| | |
|---|---|
| hsa-let-7s | hsa-miR-518f |
| hsa-miR-106a | hsa-miR-574-3p |
| hsa-miR-125a-3p | hsa-miR-628-5p |
| hsa-miR-125b | hsa-miR-888 |
| hsa-miR-130a | hsa-miR-1247 |
| hsa-miR-138 | hsa-miR-1248 |
| hsa-miR-139-3p | hsa-miR-213 |
| hsa-miR-141 | hsa-miR-338-5P |
| hsa-miR-143 | hsa-miR-378 |
| hsa-miR-193b | hsa-miR-409-3p |
| hsa-miR-218 | hsa-miR-432 |
| hsa-miR-221 | hsa-miR-497 |
| hsa-miR-320 | hsa-miR-616 |
| hsa-miR-365 | hsa-miR-663B |
| hsa-miR-429 | hsa-miR-99b* |

The overlap of miRNAs identified from follicular carcinoma (FC) and papillary carcinoma (PC) samples with t-test results having p<0.01 is shown in the Venn diagram in FIG. 23. Of the 33 miRNAs identified as being significant in the follicular carcinoma (FC) samples, 15 were only present in follicular carcinoma (FC) samples, while 18 were also found in papillary carcinoma (PC) samples. Of the 30 miRNAs in the papillary carcinoma (PC) samples, there were 12 miRNAs that were only found in the papillary carcinoma (PC) samples.

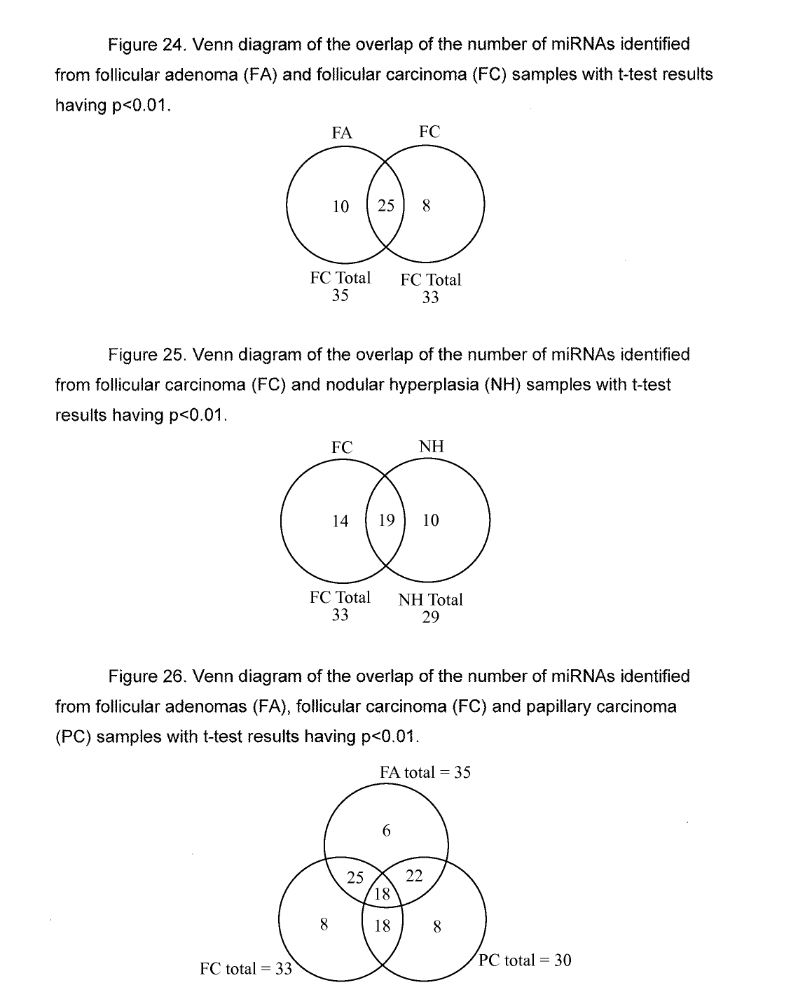

The overlap of miRNAs identified from follicular adenoma (FA) and follicular carcinoma (FC) samples with t-test results having p<0.01 is shown in the Venn diagram in FIG. 24. Of the 35 miRNAs identified as being significant in the follicular adenoma (FA), 10 were only present in follicular adenoma (FA) samples, while 25 were also found in follicular carcinoma (FC) samples. Of the 33 miRNAs in the follicular carcinoma (FC) samples, there were 8 miRNAs that were only found in the follicular carcinoma (FC) samples.

The overlap of miRNAs identified from follicular carcinoma (FC) and nodular hyperplasia (NH) sampler with t-test results having p<0.01 is shown in the Venn diagram in FIG. 25. Of the 33 miRNAs identified as being significant in the follicular carcinoma (FC) samples, 14 were only present in the follicular carcinoma (FC) samples, while 19 were also found in nodular hyperplasia (NH) samples. Of the 29 miRNAs in nodular hyperplasia (NH) samples, there were 10 miRNAs that were only found in the nodular hyperplasia (NH) samples.

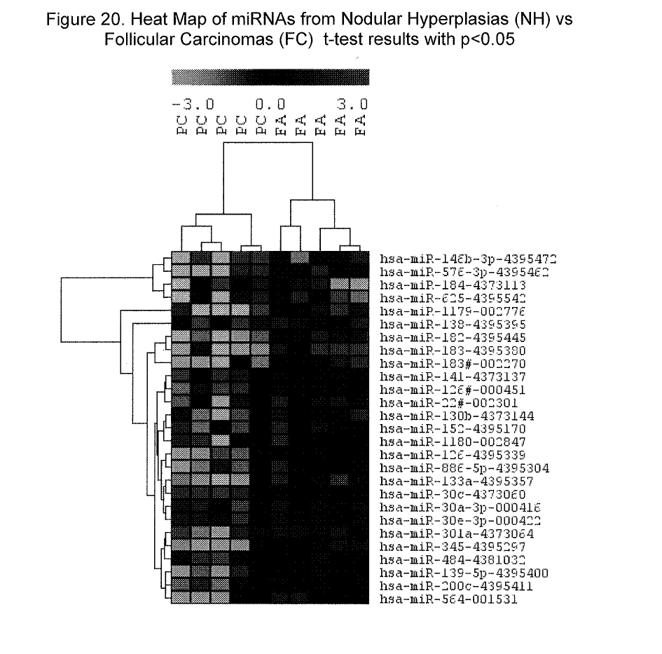

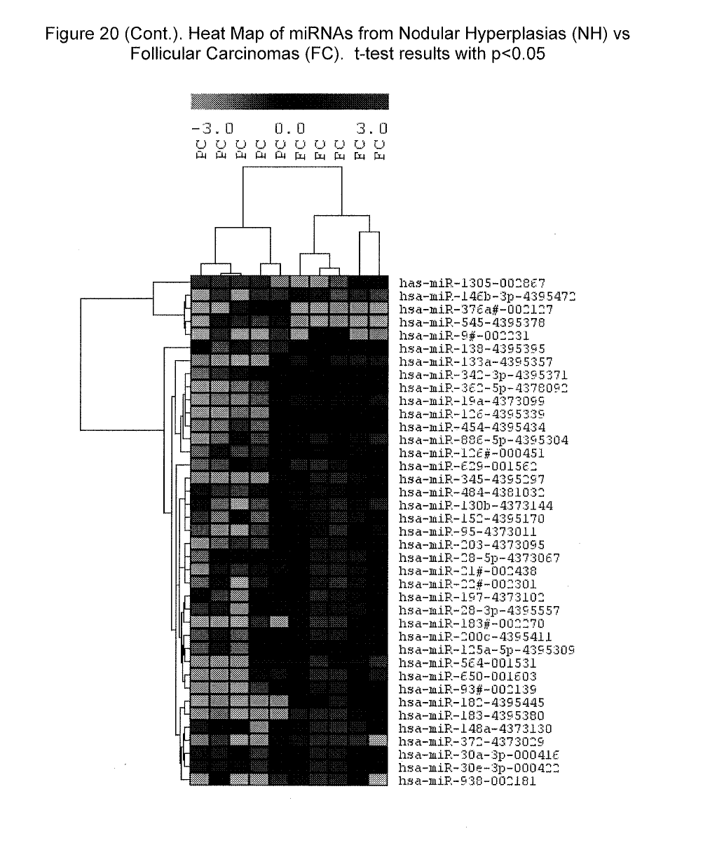

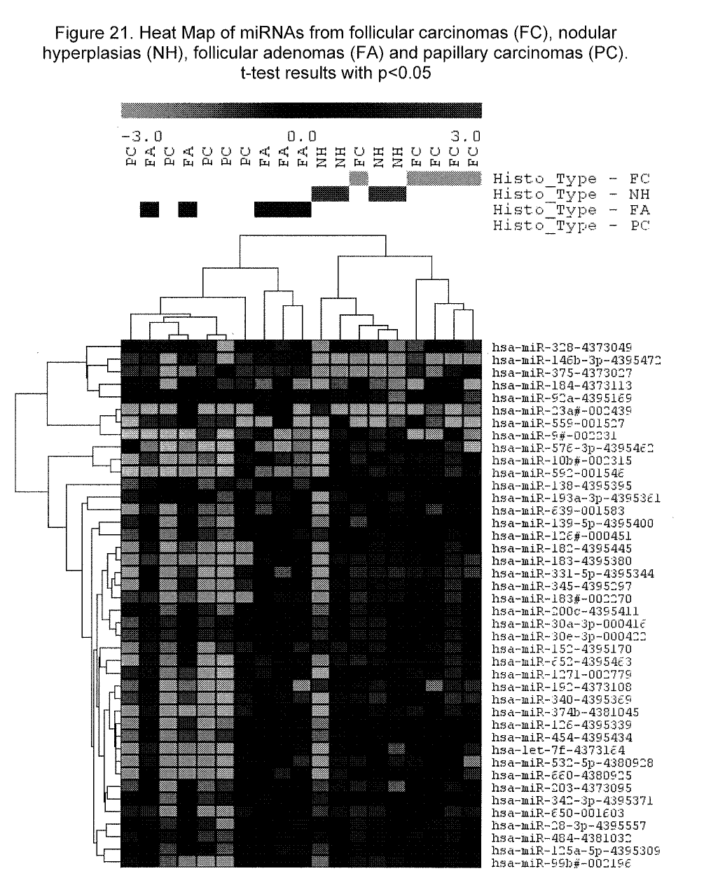

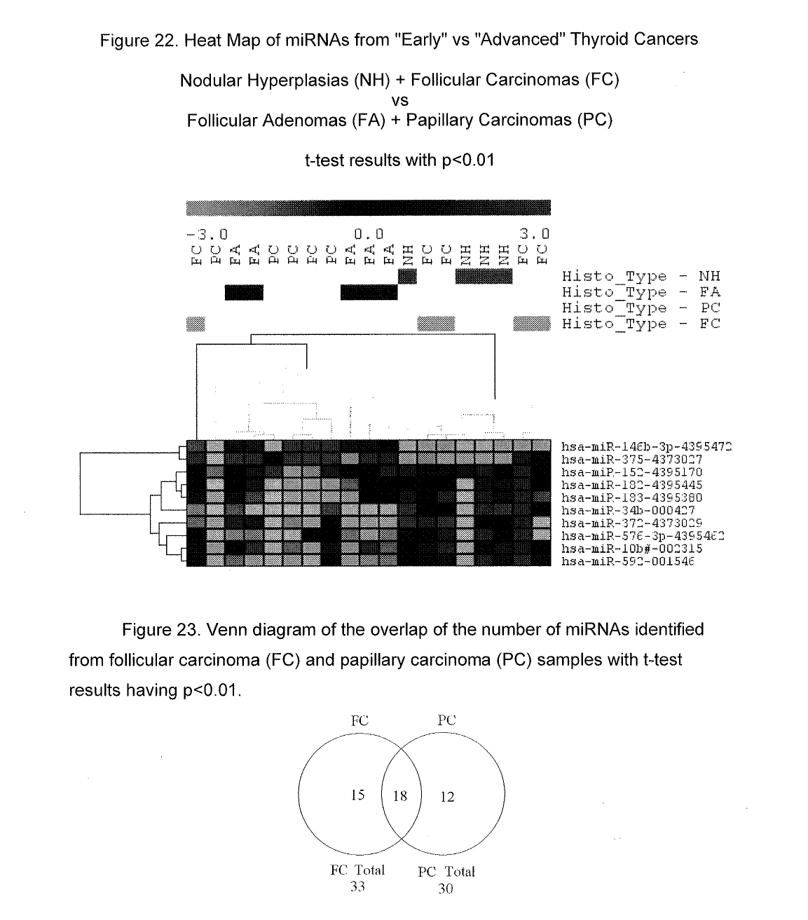

The overlap of miRNAs identified from follicular adenomas (FA), follicular carcinoma (FC) and papillary carcinoma (PC) samples with t-test results having p<0.01 is shown in the Venn diagram in FIG. 20. The distribution of the miRNAs between these three cancers is summarized in Table 5 below.

TABLE 5

| | |
|---|---|
| FA total | 35 |
| FA only | 6 |
| FA and FC | 25 |
| FA and PC | 22 |
| FC total | 33 |
| FC only | 8 |
| FC and FA | 25 |
| FC and PC | 18 |
| PC total | 30 |
| PC only | 8 |
| PC and FA | 22 |
| PC and FC | 18 |
| FA and FC and PC | 18 |

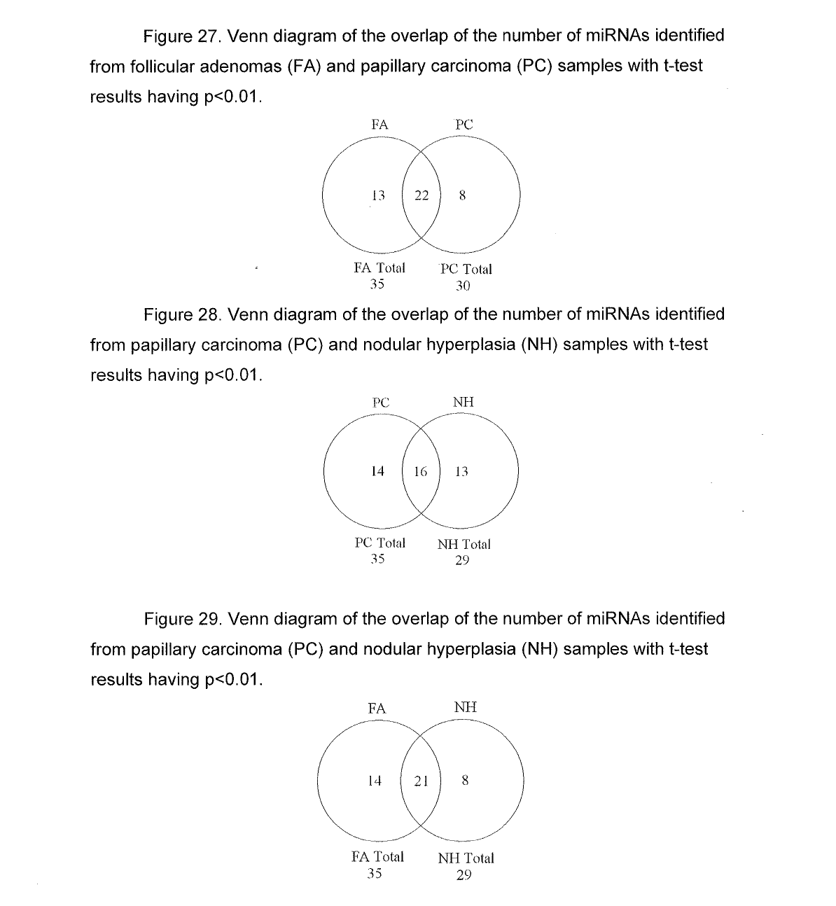

The overlap of miRNAs identified from follicular adenomas (FA) and papillary carcinoma (PC) samples with t-test results having p<0.01 is shown in the Venn diagram in FIG. 27. Of the 35 miRNAs identified as being significant in the follicular adenomas (FA) samples, 13 were only present in the adenomas (FA) samples, while 22 were also found in papillary carcinoma (PC) samples. Of the 30 miRNAs identified in the papillary carcinoma (PC) samples, there were 8 miRNAs that were only found in the papillary carcinoma (PC) samples.

The overlap of miRNAs identified from papillary carcinoma (PC) and nodular hyperplasia (NH) samples with t-test results having p<0.01 is shown in the Venn diagram in FIG. 28. Of the 30 miRNAs identified as being significant in the papillary carcinoma (PC) samples, 14 were only present in FC, while 16 were also found in nodular hyperplasia (NH) samples. Of the 29 miRNAs in nodular hyperplasia (NH) samples, there were 13 miRNAs that were only found in the nodular hyperplasia (NH) (PC) samples.

The overlap of miRNAs identified from papillary carcinoma (PC) and nodular hyperplasia (NH) samples with t-test results having p<0.01 is shown in the Venn diagram in FIG. 29. Of the 35 miRNAs identified as being significant in the papillary carcinoma (PC) samples, 14 were only present in the papillary carcinoma (PC) samples, while 21 were also found in nodular hyperplasia (NH) samples. Of the 29 miRNAs in nodular hyperplasia (NH) samples, there were 8 miRNAs that were only found in the nodular hyperplasia (NH) samples.

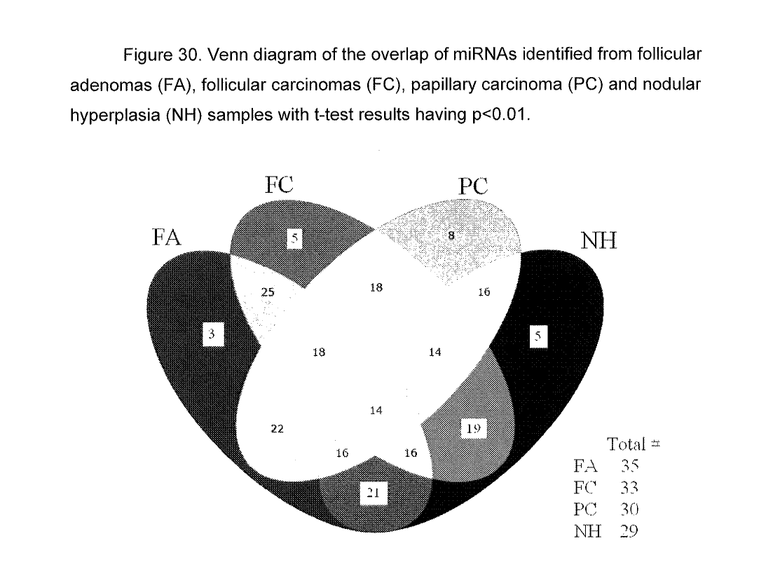

The overlap of miRNAs identified from follicular adenomas (FA), follicular carcinoma (FC), papillary carcinoma (PC) and nodular hyperplasia (NH) samples with t-test results having p<0.01 is shown in the Venn diagram in FIG. 30. The distribution of the miRNAs between these three cancers and nodular hyperplasia is summarized in Table 6 below.

TABLE 6

| Cancer | # miRNAs | Cancer | # miRNAs |
|---|---|---|---|
| FA total | 35 | FC total | 33 |
| FA only | 3 | FC only | 5 |
| FA and FC | 25 | FC and FA | 25 |
| FA and PC | 22 | FC and PC | 18 |
| FA and NH | 21 | FC and NH | 19 |
| FA, FC and PC | 18 | FC, FA and PC | 14 |
| FA, PC and NH | 16 | FC, FA and NH | 16 |
| FA, PC and NH | 16 | FC, PC and NH | 14 |
| PC total | 30 | NH total | 29 |
| PC only | 8 | NH only | 5 |
| PC and FA | 22 | NH and FA | 21 |
| PC and FC | 16 | NH and FC | 19 |
| PC and NH | 16 | NH and PC | 16 |
| PC, FA and FC | 18 | NH, FA and FC | 16 |
| PC, FA and NH | 16 | NH, FA and NH | 16 |
| PC, FC and NH | 14 | NH, FC and PC | 14 |
| FA, FC, PC and NH | 14 | | |

There are 14 miRNAs that are in common for ail four types of thyroid cancer evaluated.

A number of miRNAs are significantly differentially expressed in papillary and follicular thyroid cancer tissues when compared to normal thyroid tissue, follicular adenoma. These miRNAs together form unique “signatures” or biomarkers which can identify each tissue type. These specific miRNAs can be used to improve early diagnosis and prognosis of thyroid cancer and thereby improve treatment outcomes.

While exemplary embodiments and methods are described herein with reference to specific embodiments thereof, it will be apparent to those skilled in the art that various changes and modifications can be made, and equivalents employed, without departing from the scope of the pending claims.

1. A method of screening for thyroid cancer or a potential for developing thyroid cancer in an individual subject in need thereof, the method comprising:
a) determining expression levels of at least five miRNA selected from a first group consisting of miR-1, miR-7b, miR-26b, miR-101, miR-106a, miR-130a, miR-139, miR-141, miR-143, miR-149, miR-182, miR-190b, miR-193a, miR-193b, miR-197, miR-200c, miR-211, miR-214, miR-218, miR-302c*, miR-320, miR-324, miR-338, miR-342, miR-365, miR-367, miR-378, miR-409, miR-429, miR-432, miR-483, miR-486, miR-497, miR-518f, miR-574, miR-616, miR-628, miR-663b, miR-888, miR-1247, miR-1248, miR-1262, and miR-1305, and at least three miRNA selected from a second group Consisting of miR-21, miR-25, miR-32, miR-99b*, miR-125a, miR-125b, miR-138, miR-140, miR-181a, miR-213, miR-221, miR-222, and miR-345, in at least one sample from said individual subject;
(b) comparing the expression levels of the at least five miRNA from the first group and expression levels of the at least three miRNA from the second group with the expression levels of the corresponding miRNA established for a control individual not having thyroid cancer, and
(c) identifying differences in the miRNA expression levels between the at least one sample from said individual subject and the miRNA expression levels established for a control individual not having thyroid cancer.

2. (canceled)

3. The method of claim 1, wherein when the expression levels for the miRNA from the first group and the expression levels for the miRNA from the second group indicate an over-expression compared to the levels established for a control individual not having thyroid cancer, the method further comprises correlating the expression levels with a diagnosis of at least one of thyroid cancer and a potential for developing thyroid cancer, and optionally treating said individual subject, based on the diagnosis, by at least one of surgical removal of thyroid tissue from the individual subject and administering an effective amount of radiation and/or a chemotherapeutic agent to the individual subject.

4. The method of claim 1, wherein when the expression levels for the miRNA from the first group and the expression levels for the miRNA from the second group indicate an under-expression compared to the levels established for a control individual not having thyroid cancer, the method further comprises correlating the expression levels with a diagnosis of at least one of thyroid cancer and a potential for developing thyroid cancer, and optionally treating said individual subject, based on the diagnosis, by at least one of surgical removal of thyroid tissue from the individual subject and administering an effective amount of radiation and/or a chemotherapeutic agent to the individual subject.

5. The method of claim 1, wherein when the expression levels for the miRNA from the first group and the second group indicate an over-expression compared to the levels established for a control individual not having thyroid cancer and the expression levels for the miRNA from the first group and the second group indicate an under-expression compared to the levels established for the same miRNAs for a control individual not having thyroid cancer, the method further comprises correlating the expression levels with a diagnosis of at least one of thyroid cancer and a potential for developing thyroid cancer, and optionally treating said individual subject, based on the diagnosis, by at least one of surgical removal of thyroid tissue from the individual subject and administering an effective amount of radiation and/or a chemotherapeutic agent to the individual subject.

6. The method of claim 1, wherein the thyroid cancer is a follicular adenoma, a follicular carcinoma, a papillary carcinoma or nodular hyperplasia.

7. The method of claim 1, wherein a total of at least 5, 10, 15, 20, 25, 30, 35 or 40 miRNAs from the first group are present at levels indicating over-expression or under-expression when compared to expression levels for the same miRNAs established for a control individual not having thyroid cancer.

8. The method of claim 7, wherein a total of at least 5 miRNAs from the first and second groups have expression levels indicating over-expression or under-expression when compared to expression levels for the same miRNAs established for a control individual not having thyroid cancer.

9. The method of claim 5, wherein a total of at least 8, 10, 15, 20, 25, 30, 35 or 40 miRNAs from the first group and the second group have expression levels indicating over-expression or under-expression when compared to expression levels for the same miRNAs established for a control individual not having thyroid cancer.

10. The method of claim 5, wherein a total of at least 10 miRNAs from the first group and the second groups have expression levels indicating over-expression or under-expression when compared to expression levels for the same miRNAs established for a control individual not having thyroid cancer.

11. A method of diagnosing thyroid cancer, or a potential for developing thyroid cancer, in an individual subject in need of such diagnosis, the method comprising:
(a) determining expression levels of at least five miRNA selected from a first group consisting of miR-1, miR-7b, miR-26b, miR-101, miR-106a, miR-130a, miR-139, miR-141, miR-143, miR-149, miR-182, miR-190b, miR-193a, miR-193b, miR-197, miR-200c, miR-211, miR-214, miR-218, miR-302c*, miR-320, miR-324, miR-338, miR-342, miR-365, miR-367, miR-378, miR-409, miR-429, miR-432, miR-483, miR-486, miR-497, miR-518f, miR-574, miR-616, miR-628, miR-663b, miR-888, miR-1247, miR-1248, miR-1262, and miR-1305, and at least three miRNA from a second group consisting of miR-21, miR-25, miR-32, miR-99b*, miR-125a, miR-125b, miR-138, miR-140, miR-181a, miR-213, miR-221, miR-222, and miR-345, in at least one sample from said individual subject;
(b) comparing the expression levels of the at least five miRNA from the first group and expression levels of the at least three miRNA from a second group with expression levels of the corresponding miRNAs established for a control individual not having thyroid cancer, and
(c) identifying differences in the expression levels of the miRNAs between the individual subject and expression levels established for a control individual not having thyroid cancer.

12. A method of treating an individual subject having thyroid cancer and or a potential for developing thyroid cancer, the method comprising:
(a) determining expression levels of at least five miRNA selected from a first group consisting of miR-1, miR-7b, miR-26b, miR-101, miR-106a, miR-130a, miR-139, miR-141, miR-143, miR-149, miR-182, miR-190b, miR-193a, miR-193b, miR-197, miR-200c, miR-211, miR-214, miR-218, miR-302c*, miR-320, miR-324, miR-338, miR-342, miR-365, miR-367, miR-378, miR-409, miR-429, miR-432, miR-483, miR-486, miR-497, miR-518f, miR-574, miR-574, miR-616, miR-628, miR-663b, miR-888, miR-1247, miR-1248, miR-1262, and miR-1305, and at least three miRNA from a second group consisting of miR-21, miR-25, miR-32, miR-99b*, miR-125a, miR-125b, miR-138, miR-140, miR-181a, miR-213, miR-221, miR-222, and miR-345, in at least one sample from said individual subject;
(b) comparing the levels of the at least five miRNA from the first group and expression levels of at least three miRNA from a second group with the expression levels of the corresponding miRNAs established for a control individual not having thyroid cancer;
(c) identifying differences in the expression levels of the miRNAs between the individual subject and the expression levels established for a control individual not having thyroid cancer;
(d) correlating the expression levels with at least one of a diagnosis of thyroid cancer and a potential to develop thyroid cancer; and
(e) treating the individual subject, based on the diagnosis, by at least one of removing thyroid tissue from the individual subject and administering at least one of an effective amount of radiation and a chemotherapeutic agent to the individual subject.

13. The method of claim 12, further comprising
determining the expression levels of at least five miRNA from the first group and at least three miRNA from the second group from at least one sample obtained from said individual subject after step e).

14. The method of claim 13, further comprising
comparing the expression levels of the at least five miRNA from a first group and at least three miRNA from a second group with the expression levels of the corresponding miRNAs in the at least one sample before treating the individual subject with radiation and/or a chemotherapeutic agent and/or removing thyroid tissue from the individual subject.

15. The method of claim 14, further comprising
establishing a treatment plan based on the comparison in step.

16. The method of claim 1, wherein differential expression levels in at least miR-1, miR-21, miR-149, miR-181a, miR-2305 indicates the presence of follicular carcinoma.

17. The method of claim 1, wherein differential expression levels in miR-125a-5p, miR-182, miR-190b, miR-197, miR-200c, miR-214, miR-1262 indicates the presence of papillary carcinoma.

18. The method of claim 1, wherein differential expression levels in miR-32, miR-106a, miR-125a-3p, miR-125b, miR-130a, miR-138, miR-139-3p, miR-140-3p, miR-141, miR-143, miR-193a-3p, miR-218, miR-320, miR-378, miR-483-5p, miR-518f, miR-574-3p, and miR-628-5p indicates the presence of follicular adenoma, follicular carcinoma, papillary carcinoma.

19. The method of claim 1, wherein differential expression levels in miR-160a, miR-125a-3p, miR-125b, miR-130a, miR-138, miR-139-3p, miR-141, miR-143, miR-218, miR-221, miR-320, miR-378, miR-518f, miR-574-3p, miR-620-5p, miR-1248 indicates the presence of follicular adenoma, follicular carcinoma, and nodular hyperplasia.

20. The method of claim 1, wherein differential expression levels in miR-106a, miR-125a-3p, miR-125b, miR-130a, miR-138, miR-139-3p, miR-141, miR-143, miR-218, miR-320,miR-378, miR-518f, miR-574-3p, and miR-628-5p indicates the presence of follicular adenoma, follicular carcinoma, papillary carcinoma, and nodular hyperplasia.
